SEQUENCE LISTING (8 sequences)

SEQ ID NO: 1 - skipped

SEQ ID NO: 2 - DNA, 70 bp, synthetic construct
  source 1..70: mol_type = other DNA
aatgatacggcgaccaccgagatctacacctagcgctacactctttccctacacgacgctcttccgatct

SEQ ID NO: 3 - DNA, 66 bp, synthetic construct
  source 1..66: mol_type = other DNA
caagcagaagacggcatacgagataaccgcgggtgactggagttcagacgtgtgctcttccgatct

SEQ ID NO: 4 - DNA, 53 bp, synthetic construct
  source 1..53: mol_type = other DNA
acacgacgctcttccgatctnnnnnnnnnntgccagttaacgtcttccttctc

SEQ ID NO: 5 - DNA, 52 bp, synthetic construct
  source 1..52: mol_type = other DNA
gacgtgtgctcttccgatctnnnnnnnnnnccacacagcaaagcagaaactc

SEQ ID NO: 6 - DNA, 158 bp, synthetic construct
  source 1..158: mol_type = other DNA
tgccagttaacgtcttccttctctctctgtcatagggactctggatcccagaaggtgagaaagttaaaattcccgtcgctatcaaggaattaagagaagcaacatctccgaaagccaacaaggaaatcctcgatgtgagtttctgctttgctgtgtgg

SEQ ID NO: 7 - DNA, 218 bp, synthetic construct
  source 1..218: mol_type = other DNA
acacgacgctcttccgatctnnnnnnnnnntgccagttaacgtcttccttctctctctgtcatagggactctggatcccagaaggtgagaaagttaaaattcccgtcgctatcaaggaattaagagaagcaacatctccgaaagccaacaaggaaatcctcgatgtgagtttctgctttgctgtgtggnnnnnnnnnnagatcggaagagcacacgtc

SEQ ID NO: 8 - DNA, 314 bp, synthetic construct
  source 1..314: mol_type = other DNA
aatgatacggcgaccaccgagatctacacctagcgctacactctttccctacacgacgctcttccgatctnnnnnnnnnntgccagttaacgtcttccttctctctctgtcatagggactctggatcccagaaggtgagaaagttaaaattcccgtcgctatcaaggaattaagagaagcaacatctccgaaagccaacaaggaaatcctcgatgtgagtttctgctttgctgtgtggnnnnnnnnnnagatcggaagagcacacgtctgaactccagtcacccgcggttatctcgtatgccgtcttctgcttg

(19) United States
(12) Patent Application Publication
(10) Pub. No.: US 2026/0209851 A1
(43) Pub. Date: Jul. 23, 2026

(54) METHOD FOR EFFICIENT MULTIPLEX DETECTION AND QUANTIFICATION OF GENETIC ALTERATIONS

(71) Applicant: Lucence Life Sciences Pte. Ltd., Singapore (SG)

(72) Inventors: Jonathan POH, Singapore (SG); Kao Chin NGEOW, Singapore (SG); Yukti CHOUDHURY, Singapore (SG); Min-Han TAN, Singapore (SG)

(21) Appl. No.: 19/135,002

(22) PCT Filed: Dec. 2, 2022
(86) PCT No.: PCT/SG2022/050881

§ 371 Date: Jun. 2, 2025

(51) Int. Cl. C12Q 1/6883; C12N 15/10; C12Q 1/6806; C12Q 1/686; C12Q 1/6886

(52) U.S. Cl. CPC C12Q 1/6883; C12N 15/1065; C12Q 1/6806; C12Q 1/686; C12Q 1/6886; C12Q 2600/156; C12Q 2600/16

(57) ABSTRACT

Disclosed is a method of using highly multiplexed amplicon-based target sequencing to detect genomic alterations in nucleic acids within a biological sample. Also disclosed is a kit for detecting genomic alterations within a biological sample.

### FIELD OF INVENTION

The present disclosure relates to the use of highly multiplexed amplicon-based target sequencing to detect genomic alterations in genetic material isolated from biological samples. In particular, the present invention relates to the detection of genomic alterations in nucleic acids present in biological liquid biopsies and other biological samples.

### BACKGROUND

Highly multiplexed amplicon-based target sequencing offers a highly scalable, sensitive approach for the detection of genomic alterations and are applicable to the detection of rare alterations in genetic material (DNA/RNA) isolated from liquid biopsies and other sample types (cellular, tissue, plasma, etc). One of the gold standard methods for high-throughput detection and quantification of rare genetic events is the next-generation sequencing (NGS)-based detection methods. Conventional NGS-based methods are characterized by an error rate of 0.1-1%, with every 1 of 100 or 1000 bases being called incorrectly due to artifacts introduced during sample preparation and sequencing. An inevitable drawback of such conventional high-throughput detection methodologies is the need for repeated sampling or deep sequencing of a large number of molecules, that may not be readily possible due to limitations of sample input amount. This limitation is especially apparent during liquid biopsies. To overcome limitations of sample input, the person skilled in the art typically would have to amplify the nucleic acid sequences present in the sample. However, it is known that traditional amplification methods lack reliability and accuracy, which are required for the detection of rare genomic alterations that occur at extremely low frequencies (i.e., <1%) in the background of otherwise unchanged nucleic acid sequences.

The incorporation of unique barcode sequences during DNA target capture in conventional NGS-based methods enables an approximate 100-fold error correction as compared to standard NGS-based methods. By grouping families of amplicons possessing identical barcode sequences, errors propagated during the sequencing process, which appear only in a subset of family members, can be differentiated from true mutations, which appear in all family members. This vast improvement in error detection greatly increases the sensitivity of NGS-based methods, enabling their use in liquid biopsy, where the fraction of circulating tumor DNA (ctDNA) in cell-free DNA (cfDNA) is often low. However, a drawback of such barcode-based methods is the need for redundant sequencing to achieve adequate family sizes, thus inflating the overall cost of sequencing.

Liquid biopsies require ultradeep sequencing of a small amount of starting genetic material, which typically has quantities limited by biology, to achieve highly sensitive detection of genetic alterations. This is of particular importance for cancer patients with low tumour burden, and in the context of early cancer detection and screening for the wider population. While targeted sequencing of rationally curated regions allows for this to be economically feasible, the sheer spectrum of cancer-related genetic alterations across different cancer types translates to a need for a method of target capture that is scalable while retaining a high level of efficiency. Achieving a Goldilocks balance of high sensitivity from limited starting material, high specificity/low noise, high scalability and low sequencing costs is a non-trivial challenge.

As such, there is a need to provide a method for sensitive and accurate detection of genomic alterations that overcomes, or at least ameliorates, one or more of the disadvantages described above. There is a need to provide a method to minimise inflation of consensus coverage arising due to barcode duplication or resampling in existing barcode-based methods to thereby allow for sequencing at much lower read depths to achieve similar sensitivities and improving the cost-effectiveness of the overall method. There is a need to provide a method to accurately detect genomic alterations from a small amount of starting biological sample comprising a small amount of genetic material.

### SUMMARY

In a first aspect, the present disclosure refers to a method of detecting genomic alterations within a biological sample comprising nucleic acids, comprising the steps of:
(a) extracting nucleic acid from the biological sample;
(b) performing a plurality of multiplexed PCR reactions on the extracted nucleic acid using:
   a plurality of target capture primer pairs specific to a plurality of target genes that are capable of undergoing genomic alteration,
   wherein each target capture primer pair comprises a forward target capture primer and a reverse target capture primer specific to a target gene that is capable of undergoing genomic alteration,
   wherein each forward target capture primer and each reverse target capture primer comprises a target-specific sequence at its 3′ end that is specific to the target gene, a barcode sequence linker, and an adapter-specific sequence at its 5′ end,
   thereby generating a plurality of amplicons comprising a plurality of target gene sequences corresponding to the plurality of target genes that are capable of undergoing genomic alteration;
(c) removing target capture primers that have not been incorporated into the plurality of amplicons generated in step (b) using at least one nuclease, thereby generating a purified mixture of the plurality of amplicons comprising the plurality of target gene sequences;
(d) amplifying the plurality of amplicons in the purified mixture obtained from step (c) by using universal indexed adapter primers to generate a sequencing library, wherein each amplicon of the sequencing library comprises two barcode sequences;
(e) purifying the sequencing library obtained from step (d);
(f) subjecting the purified sequencing library from step (e) to multiplex sequencing on a next-generation sequencing platform to obtain a plurality of sequencing reads;
(g) mapping the plurality of sequencing reads obtained from step (f) to a first reference genome;
(h) grouping the sequencing reads where the barcode sequences of the sequencing reads are identical into a consensus cluster;
(i) performing a sequence alignment of each consensus cluster obtained from step (h) with all consensus clusters having at least one overlapping barcode sequence, then determining the presence of a consensus base in each sequence alignment result to generate a consensus sequencing read;
(j) mapping each of the consensus sequencing read from step (i) with a second reference genome;
(k) identifying the differences between the sequencing read and the reference genome from step j) to thereby determine the presence of genomic alteration in the nucleic acid present in the biological sample.

In a second aspect, the present disclosure refers to a kit for detecting genomic alterations within a biological sample according to the method disclosed herein, comprising a plurality of target capture primer pairs specific to a plurality of target genes that are capable of undergoing genomic alteration as defined in step (b) of the first aspect, and instructions for use in the method disclosed herein.

### BRIEF DESCRIPTION OF DRAWINGS

The invention will be better understood with reference to the detailed description when considered in conjunction with the non-limiting examples and the accompanying drawings, in which:

FIG. 1 is a general overview of the experimental workflow of the NGS library preparation step of the method described herein. The NGS library preparation step can be categorized into three major steps (multiplex target capture PCR step, followed by the exonuclease treatment step, and finally the indexing PCR step).

FIG. 2 (comprised of FIGS. 2A and 2B) illustrates examples of the structure of a target capture primer and the structure of a final library amplicon containing dual barcodes/unique molecular identifiers (UMIs). FIG. 2A illustrates an example of a target capture primer structure comprising three regions; a partial Illumina sequencing adapter on the 5′ end, a target-specific sequence of length 17 to 40 nucleotides on the 3′ end to enable hybridisation with target DNA, and a linking barcode sequence composed of 10 random nucleotides. FIG. 2B illustrates a final library amplicon structure containing dual barcodes/UMIs required for sequencing on the Illumina platform.

FIG. 3 (comprised of FIGS. 3A and 3B) is an overview of the dual barcode-subgraph consensus clustering workflow. FIG. 3A illustrates the workflow of the PCR amplification step. During PCR amplification with barcoded primers, a single target DNA molecule may be tagged by multiple unique barcode sequences, if a different barcoded primer anneals to and extends the target DNA molecule in each cycle. This is referred to as barcode duplication. Given the exponential nature of PCR, under theoretical conditions, barcode duplication proceeds at an exponential rate with increasing PCR cycles. Only complete libraries with barcodes and adapters on both ends are sequenceable (grey arrows). Each of F1-7 and R1-7 refers to a unique barcode sequence. FIG. 3B illustrates the workflow of the barcode clustering step. Clustering of libraries with at least one overlapping barcode (either F or R) minimises barcode duplication. When two PCR cycles are performed, two distinct barcode combinations are generated from a double-stranded DNA molecule (no duplication). When three PCR cycles are performed, eight distinct barcode combinations can be generated from a double-stranded DNA molecule, representing a four-fold barcode duplication rate. Application of subgraph consensus clustering enables clustering of these eight barcode combinations into four clusters, reducing the barcode duplication rate by two-fold.

FIG. 4 (comprised of FIGS. 4A, 4B, 4C and 4D) illustrates the comparative Tapestation 4200 high sensitivity D1000 screentape profiles of libraries subjected to exonuclease treatment or without exonuclease treatment for an increasing number of amplicons in exemplary panels. FIG. 4A demonstrates an unsuccessful library from a workflow without exonuclease treatment and target capture primers representing 545 amplicons. FIG. 4B demonstrates successful libraries containing a high proportion of specific products generated from a workflow incorporating exonuclease treatment and target capture primers representing 334 amplicons. FIG. 4C demonstrates successful libraries containing a high proportion of specific products generated from a workflow incorporating exonuclease treatment and target capture primers representing 1167 amplicons. FIG. 4D demonstrates successful libraries containing a high proportion of specific products generated from a workflow incorporating exonuclease treatment and target capture primers representing 2624 amplicons.

FIG. 5 (comprised of FIGS. 5A and 5B) shows the comparison of error rates and required sequencing depth between using the subgraph consensus clustering workflow and a standard barcode clustering workflow. 65 cfDNA samples (2.5 ng) were sequenced and analysed using both the subgraph consensus clustering workflow and a standard barcode clustering workflow. FIG. 5A shows that subgraph consensus clustering method reduces error rates (i.e., mean number of noise variants) compared to using standard barcode clustering workflow. FIG. 5B shows that subgraph consensus clustering reduces the required sequencing depth compared to using standard barcode clustering workflow.

FIG. 6 shows that subgraph consensus clustering workflow retained high sensitivity of the method as described herein at reduced sequencing depths. Libraries from two replicates of the Horizon Discovery™ Multiplex I cfDNA Reference Standard (HD779) were down-sampled to 2×, 4×, 6×, 8×, and 10× reduced sequencing read depths and analysed using a standard barcode clustering workflow and the subgraph consensus clustering workflow. Sensitivity was calculated as the number of variants detected/total number of variants (8 genomic alterations in total) in the HD779 Reference Standard.

FIG. 7 illustrates the average on-target template capture rate enumerated based on unique barcodes in a 545-amplicon panel. Assuming 330 copies of DNA template are available for target capture per ng of DNA input, the majority of available template had failed to be captured with just two PCR cycles. Increasing cycle number increased rate of target capture.

### DETAILED DESCRIPTION

The present disclosure describes a method of amplicon-based target capture that involves the incorporation of barcode sequences in both the forward and reverse primers during initial DNA target capture. This allows information to be captured from both strands of DNA in the double-stranded DNA input, an approach that has not previously been adopted in amplicon-based target capture. The overall doubling of DNA target capture is therefore expected to improve the sensitivity of the method compared to conventional amplicon-based target capture methods which target capture is only performed on one DNA strand of the double-stranded DNA source material.

In a first aspect, the present disclosure refers to a method of detecting genomic alterations within a biological sample comprising nucleic acids, comprising the steps of:
(a) extracting nucleic acid from the biological sample;
(b) performing a plurality of multiplexed PCR reactions on the extracted nucleic acid using:
   a plurality of target capture primer pairs specific to a plurality of target genes that are capable of undergoing genomic alteration,
   wherein each target capture primer pair comprises a forward target capture primer and a reverse target capture primer specific to a target gene that is capable of undergoing genomic alteration,
   wherein each forward target capture primer and each reverse target capture primer comprises a target-specific sequence at its 3′ end that is specific to the target gene, a barcode sequence linker, and an adapter-specific sequence at its 5′ end,
   thereby generating a plurality of amplicons comprising a plurality of target gene sequences corresponding to the plurality of target genes that are capable of undergoing genomic alteration;
(c) removing target capture primers that have not been incorporated into the plurality of amplicons generated in step (b) using at least one nuclease, thereby generating a purified mixture of the plurality of amplicons comprising the plurality of target gene sequences;
(d) amplifying the plurality of amplicons in the purified mixture obtained from step (c) by using universal indexed adapter primers to generate a sequencing library, wherein each amplicon of the sequencing library comprises two barcode sequences;
(e) purifying the sequencing library obtained from step (d);
(f) subjecting the purified sequencing library from step (e) to multiplex sequencing on a next-generation sequencing platform to obtain a plurality of sequencing reads;
(g) mapping the plurality of sequencing reads obtained from step (f) to a first reference genome;
(h) grouping the sequencing reads where the barcode sequences of the sequencing reads are identical into a consensus cluster;
(i) performing a sequence alignment of each consensus cluster obtained from step (h) with all consensus clusters having at least one overlapping barcode sequence, then determining the presence of a consensus base in each sequence alignment result to generate a consensus sequencing read;
(j) mapping each of the consensus sequencing read from step (i) with a second reference genome;
(k) identifying the differences between the consensus sequence read and the reference genome from step (j) to thereby determine the presence of genomic alteration in the nucleic acid present in the biological sample.

In one example, the genomic alteration is selected from the group consisting of single-nucleotide variations, insertions (such as duplication), deletions, genomic copy number alterations, deletions of homopolymeric regions, total mutation (or variant) load, detection of microbial nucleic acid sequences, detection of polymorphisms or single-nucleotide variations in nucleic acid sequences, and structural rearrangements in DNA giving rise to variant RNA molecules. In one example, “single nucleotide variations” refer to variation in a single nucleotide that occurs at a specific position in the genome, differing from the nucleotide defining the position in the reference genome. In one example, the “insertion” is a sequence change where at least one nucleotide is inserted between two nucleotides. In one example, the “insertion” is a sequence change where more than 10 nucleotides are inserted between two nucleotides. In one example, the “insertion” is a sequence change where more than 20 nucleotides are inserted between two nucleotides. In one example, the “insertion” is a sequence change where more than 30 nucleotides are inserted between two nucleotides. In one example, the “insertion” is a sequence change where more than 40 nucleotides are inserted between two nucleotides. In one example, the “insertion” is a sequence change where more than 50 nucleotides are inserted between two nucleotides. In one example, the “insertion” may be a “small insertion” where less than 50 nucleotides are inserted between two nucleotides. In one example, the “insertion” is a “duplication”. In one example, the “duplication” is a sequence change where a copy of one or more nucleotides are inserted directly 3′-flanking of the original copy. In one example, the “deletion” is a sequence change where at least one nucleotide is removed. In one example, the “deletion” is a sequence change where more than 10 nucleotides are removed. In one example, the “deletion” is a sequence change where more than 20 nucleotides are removed. In one example, the “deletion” is a sequence change where more than 30 nucleotides are removed. In one example, the “deletion” is a sequence change where more than 40 nucleotides are removed. In one example, the “deletion” is a sequence change where more than 50 nucleotides are removed. In one example, the “deletion” may be a “small deletion” where less than 50 nucleotides are removed. In one example, the term “copy number alteration” refers to the repetition of sections of the genome (duplication) or loss of sections of the genome (deletion). In one example, the term “deletions of homopolymeric regions” refers to the shortening of a homopolymeric tracts in the genome. An example of “deletions of homopolymeric region” is GCGAAAAAAAAAAAAAAATA becomes GCGAAATA, this a deletion of 12 A's from the homopolymeric tract of 15 A's. In one example, the term “polymorphism” refers to a variation in a single nucleotide that occurs at a specific position in the genome, and is a variation in all copies of the organism's genome, differing from nucleotide defining the position in the organism's population (reference). A person skilled in the art is aware that the sum of all of the variants within the nucleic acid sequence is known as total mutation (or variant) load or tumour mutational burden (TMB). A person skilled in the art is also aware that determining the total mutation (or variant) load or tumour mutational burden (TMB) is useful in determining the therapeutic target of certain diseases (such as cancer). Microbial nucleic acid sequences are considered non-human genomic sequences and/or foreign DNA sequences. In one example, the microbial nucleic acid sequence is microbial DNA sequence. In one example, the microbial nucleic acid sequence is microbial RNA sequence. In one example, the microbial RNA sequence is SARS-CoV-2 RNA sequence. In one example, the structural rearrangement may be structural rearrangement(s) in the DNA giving rise to variant RNA molecules. In one example, the structural rearrangement may be structural variants of RNA molecules. In one example, the structural rearrangement may be copy number alterations in the DNA, giving rise to variable amounts of RNA molecules. In one example, the structural rearrangements may be structural variants of RNA molecules and copy number alterations of RNA molecules. In one example, the structural arrangement may be structural rearrangement(s) in DNA molecules. In one example, the term “rearrangement” refers to rearrangement in the order of sections of the DNA, giving rise to a variant transcript of an RNA molecule. In one example, the structural rearrangement is a fusion, such as a gene fusion. In one example, the term “fusion” refers to structural variations produced through structural rearrangements, such as interchromosomal or intrachromosomal rearrangements. In one example, the structural rearrangement may include, but are not limited to, deletion, insertion (such as duplication), inversion, transversion, translocation, alternative splicing, and the like. In one example, the term “translocation” refers to rearrangement of parts between non-homologous chromosomes, which can result in “fusion”. In one example, “altered splicing” refers to aberrant splicing of a single gene transcript that may cause one or more exons in sequence to be spliced out of the RNA, bringing usually more distant exons of the same gene in juxtaposition. Altered splicing involves the same gene, compared to fusion which is a definition reserved for two genes. One example of altered splicing includes MET exon 14 skipping where exon 14 of MET gene is spliced out bringing exon 13 and exon 15 in proximity. In one example, the genomic alteration may be RNA structural variants. In another example, the genomic alteration may be copy number alterations of RNA molecules.

In one example, the nucleic acid is selected from the group consisting of DNA and RNA. In one example, the DNA is selected from the group consisting of genomic DNA, tumour tissue DNA, circular DNA, cell-free DNA (cfDNA) and circulating tumor DNA (ctDNA). In one example, the DNA is ctDNA. In one example, the DNA is cfDNA. In one example, the RNA is selected from the group consisting of messenger RNA, circular RNA and non-coding RNA. In one example, the method disclosed herein uses DNA as the nucleic acid input. In one example, the method disclosed herein uses RNA as the nucleic acid input. In one example, RNA can be converted into DNA prior to step (b) of the method of the first aspect.

In one example, the nucleic acid is present freely in the biological sample. In one example, the nucleic acid is originally encapsulated within cells and needs to be extracted as defined in step (a) of the method of the first aspect. In one example, the cell may be any type of cell in the body. In one example, the cell is from bone, epithelial, cartilage, adipose tissue, nerves, muscle, connective tissue, esophagus, stomach, liver, gallbladder, pancreas, adrenal glands, bladder, gallbladder, large intestine, small intestine, kidneys, liver, pancreas, colon, stomach, thymus, spleen, brain, spinal cord, heart, lungs, eyes, corneal, skin, or islet tissue or organs. In one example, the cell may be a cancer cell, a stem cell, an endothelial cell, or a fat cell. In one example, the cell is a blood cell. The blood cell may be a white blood cell, or a platelet. In one example, the cell is selected from cancer cells known to harbour genomic alterations. Various methods for nucleic acid extraction are known in the art and may be used for the purpose of the disclosed method. In one example, the nucleic acid is extracted from the biological sample using a kit such as, but not limited to QIAamp Circulating Nucleic Acid kit (Qiagen), Zymo Quick-cfRNA Serum & Plasma Kit (Zymo Research), NextPrep™ Magnazol™ cfRNA Isolation Kit (PerkinElmer), Isopure Plasma cfDNA/RNA Isolation Kit (Aline Biosciences), QIAamp ccfDNA/RNA Kit (Qiagen), MagMAX™ Cell-Free Total Nucleic Acid Isolation Kit (Applied Biosystems), DNeasy Blood & Tissue Kit (Qiagen), Wizard Genomic DNA Purification Kit (Promega), PureLink Genomic DNA Mini Kit (Invitrogen), etc.

In one example, the biological sample comprising nucleic acids is selected from the group consisting of a liquid sample, a tissue sample, and a cell sample. In one example, the liquid sample is a bodily fluid. In one example, the bodily fluid is selected from the group consisting of blood, bone marrow, cerebral spinal fluid, peritoneal fluid, pleural fluid, lymph fluid, ascites, serous fluid, sputum, lacrimal fluid, stool, urine, saliva, ductal fluid from breast, gastric juice and pancreatic juice. In one example, the bodily fluid is blood. In another example, the blood is plasma.

In one example, the amount of nucleic acid within the biological sample is from 1 ng to 300 ng, or from 10 ng to 290 ng, or from 20 ng to 280 ng, or from 30 ng to 270 ng, or from 40 ng to 260 ng, or from 50 ng to 250 ng, or from 60 ng to 240 ng, or from 70 ng to 230 ng, or from 80 ng to 220 ng, or from 90 ng to 210 ng, or from 100 ng to 200 ng, or from 110 ng to 190 ng, or from 120 ng to 180 ng, or from 130 ng to 170 ng, or from 140 ng to 160 ng, or about 1 ng, or about 2 ng, or about 2.5 ng, or about 5 ng, or about 10 ng, or about 20 ng, or about 30 ng, or about 40 ng, or about 50 ng, or about 60 ng, or about 70 ng, or about 80 ng, or about 90 ng, or about 100 ng, or about 110 ng, or about 120 ng, or about 130 ng, or about 140 ng, or about 150 ng, or about 160 ng, or about 170 ng, or about 180 ng, or about 190 ng, or about 200 ng, or about 210 ng, or about 220 ng, or about 230 ng, or about 240 ng, or about 250 ng, or about 260 ng, or about 270 ng, or about 280 ng, or about 290 ng, or about 300 ng.

In one example, the method of the present disclosure comprises performing a plurality of multiplexed PCR reactions on the extracted nucleic acid as defined in step (b) of the method of the first aspect. In one example, the target DNA is captured by PCR using a highly multiplexed pool of target capture primers. In one example, the method of the present disclosure comprises performing a plurality of multiplexed PCR reactions on the extracted nucleic acid using:
a plurality of target capture primer pairs specific to a plurality of target genes that are capable of undergoing genomic alteration,
wherein each target capture primer pair comprises a forward target capture primer and a reverse target capture primer specific to a target gene that is capable of undergoing genomic alteration,
wherein each forward target capture primer and each reverse target capture primer comprises a target-specific sequence at its 3′ end that is specific to the target gene, a barcode sequence linker, and an adapter-specific sequence at its 5′ end,
thereby generating a plurality of amplicons comprising a plurality of target gene sequences corresponding to the plurality of target genes that are capable of undergoing genomic alteration.

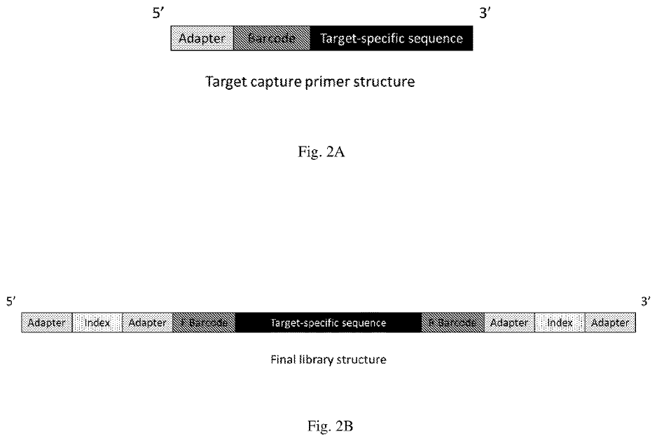

In one example, each forward target capture primer comprises three regions; a partial Illumina sequencing adapter on the 5′ end, a target-specific sequence comprising 15 to 70 nucleotides on the 3′ end to enable hybridisation with target DNA, and a linking barcode sequence comprised of 10 random nucleotides (FIG. 2A). In one example, each reverse target capture primer comprises three regions; a partial Illumina sequencing adapter on the 5′ end, a target-specific sequence comprising 15 to 70 nucleotides on the 3′ end to enable hybridisation with target DNA, and a linking barcode sequence comprised of 10 random nucleotides (FIG. 2A).

In one example, the length of the target-specific sequence is from 15 nucleotides to 70 nucleotides, or from 16 nucleotides to 69 nucleotides, or from 17 nucleotides to 68 nucleotides, or from 18 nucleotides to 67 nucleotides, or from 19 nucleotides to 66 nucleotides, or from 20 nucleotides to 65 nucleotides, or from 21 nucleotides to 64 nucleotides, or from 22 nucleotides to 63 nucleotides, or from 23 nucleotides to 62 nucleotides, or from 24 nucleotides to 61 nucleotides, or from 25 nucleotides to 60 nucleotides, or from 26 nucleotides to 59 nucleotides, or from 27 nucleotides to 58 nucleotides, or from 28 nucleotides to 57 nucleotides, or from 29 nucleotides to 56 nucleotides, or from 30 nucleotides to 55 nucleotides, or from 31 nucleotides to 54 nucleotides, or from 32 nucleotides to 53 nucleotides, or from 33 nucleotides to 52 nucleotides, or from 34 nucleotides to 51 nucleotides, or from 35 nucleotides to 50 nucleotides, or from 36 nucleotides to 49 nucleotides, or from 37 nucleotides to 48 nucleotides, or from 38 nucleotides to 47 nucleotides, or from 39 nucleotides to 46 nucleotides, or from 40 nucleotides to 45 nucleotides, or from 41 nucleotides to 44 nucleotides, or 15 nucleotides, or 16 nucleotides, or 17 nucleotides, or 18 nucleotides, or 19 nucleotides, or 20 nucleotides, or 21 nucleotides, or 22 nucleotides, or 23 nucleotides, or 24 nucleotides, or 25 nucleotides, or 26 nucleotides, or 27 nucleotides, or 28 nucleotides, or 29 nucleotides, or 30 nucleotides, or 31 nucleotides, or 32 nucleotides, or 33 nucleotides, or 34 nucleotides, or 35 nucleotides, or 36 nucleotides, or 37 nucleotides, or 38 nucleotides, or 39 nucleotides, or 40 nucleotides, or 41 nucleotides, or 42 nucleotides, or 43 nucleotides, or 44 nucleotides, or 45 nucleotides, or 46 nucleotides, or 47 nucleotides, or 48 nucleotides, or 49 nucleotides, or 50 nucleotides, or 51 nucleotides, or 52 nucleotides, or 53 nucleotides, or 54 nucleotides, or 55 nucleotides, or 56 nucleotides, or 57 nucleotides, or 58 nucleotides, or 59 nucleotides, or 60 nucleotides, or 61 nucleotides, or 62 nucleotides, or 63 nucleotides, or 64 nucleotides, or 65 nucleotides, or 66 nucleotides, or 67 nucleotides, or 68 nucleotides, or 69 nucleotides, or 70 nucleotides. In one example, the length of the target-specific sequence is from 17 nucleotides to 40 nucleotides.

In one example, the barcode sequence is an oligonucleotide comprising 8 to 30 random nucleotides. In one example, the barcode sequence is an oligonucleotide comprising 9 to 29 random nucleotides. In one example, the barcode sequence is an oligonucleotide comprising 10 to 28 random nucleotides. In one example, the barcode sequence is an oligonucleotide comprising 8 random nucleotides. In one example, the barcode sequence is an oligonucleotide comprising 9 random nucleotides. In one example, the barcode sequence is an oligonucleotide comprising 10 random nucleotides. In one example, the barcode sequence is an oligonucleotide comprising 11 random nucleotides. In one example, the barcode sequence is an oligonucleotide comprising 12 random nucleotides. In one example, the barcode sequence is an oligonucleotide comprising 13 random nucleotides. In one example, the barcode sequence is an oligonucleotide comprising 14 random nucleotides. In one example, the barcode sequence is an oligonucleotide comprising 15 random nucleotides. In one example, the barcode sequence is an oligonucleotide comprising 16 random nucleotides. In one example, the barcode sequence is an oligonucleotide comprising 17 random nucleotides. In one example, the barcode sequence is an oligonucleotide comprising 18 random nucleotides. In one example, the barcode sequence is an oligonucleotide comprising 19 random nucleotides. In one example, the barcode sequence is an oligonucleotide comprising 20 random nucleotides. In one example, the barcode sequence is an oligonucleotide comprising 21 random nucleotides. In one example, the barcode sequence is an oligonucleotide comprising 22 random nucleotides. In one example, the barcode sequence is an oligonucleotide comprising 23 random nucleotides. In one example, the barcode sequence is an oligonucleotide comprising 24 random nucleotides. In one example, the barcode sequence is an oligonucleotide comprising 25 random nucleotides. In one example, the barcode sequence is an oligonucleotide comprising 26 random nucleotides. In one example, the barcode sequence is an oligonucleotide comprising 27 random nucleotides. In one example, the barcode sequence is an oligonucleotide comprising 28 random nucleotides. In one example, the barcode sequence is an oligonucleotide comprising 29 random nucleotides. In one example, the barcode sequence is an oligonucleotide comprising 30 random nucleotides. In one specific example, the barcode sequence is an oligonucleotide comprising 10 random nucleotides which can be represented as NNNNNNNNNN (SEQ ID NO: 1).

In one example, the plurality of multiplexed PCR reactions performed on the nucleic acid in step (b) of the method of the first aspect comprises a first PCR step comprising 3 to 15 PCR cycles and the amplification of the plurality of amplicons in the purified mixture as defined in step (d) of the method of the first aspect comprises a second PCR step comprising 4 to 32 PCR cycles. In one example, the plurality of multiplexed PCR reactions performed on the nucleic acid in step (b) of the method of the first aspect comprises a first PCR step. In one example, the first PCR comprises a low number of PCR cycles for target capture and limited amplification. In one example, the plurality of multiplexed PCR reactions performed on the nucleic acid in step (b) of the method of the first aspect comprises a first PCR step comprising 3 to 15 PCR cycles. In one specific example, the first PCR step comprises 3 to 5 PCR cycles. In one example, the amplification of the plurality of amplicons in the purified mixture as defined in step (d) of the method of the first aspect comprises a second PCR step. In one example, the amplification of the plurality of amplicons in the purified mixture as defined in step (d) of the method of the first aspect comprises a second PCR step comprising 4 to 32 PCR cycles. In another specific example, the second PCR step comprises 14 to 16 PCR cycles. In one example, the plurality of amplicons in the purified mixture as defined in step (d) of the method of the first aspect is subjected to an indexing PCR step (i.e., the second PCR step) where further DNA amplification occurs to increase yields for sequencing, and where predetermined index sequences are added to each amplicon to facilitate subsequent post-sequencing demultiplexing. In one example, the number of PCR cycles in the first PCR step and the second PCR step are independent of each other. In another example, the first PCR step comprises 3 to 5 PCR cycles and the second PCR step comprises 14 to 16 PCR cycles.

In one example, the method of the present disclosure comprises removing target capture primers that have not been incorporated into the plurality of amplicons generated in step (b) of the method of the first aspect using at least one enzyme capable of removing unincorporated target capture primers, for example by degrading the primers via cleavage of phosphodiester bonds between nucleotides of the primers, to thereby generate a purified mixture of the plurality of amplicons comprising the plurality of target gene sequences. In one example, the enzyme is a nuclease. In one example, the method of the present disclosure comprises removing target capture primers that have not been incorporated into the plurality of amplicons generated in step (b) of the method of the first aspect using at least one nuclease, thereby generating a purified mixture of the plurality of amplicons comprising the plurality of target gene sequences. In one example, the nuclease is an endonuclease. In one example, the nuclease is an exonuclease. In one example, the target capture primers to be removed are single-stranded DNA sequences. In one example, the target capture primers are single-stranded DNA primers. In one example, the target capture primers are excess unincorporated single-stranded DNA primers. In one example, the target capture primers are excess single-stranded DNA primers, which remain unincorporated after the target capture step of the multiplexed PCR reactions as defined in step (b) of the first aspect. In one example, the exonuclease may be a single-stranded RNA specific exonuclease.

In one example, the target capture primers that have not been incorporated into amplicons are removed in step (c) of the method of the first aspect using nucleases selected from the group consisting of exonuclease I, exonuclease T, exonuclease VII, mung bean nuclease (an endonuclease), nuclease P1 (an endonuclease) and nuclease S1 (an endonuclease). In one example, the target capture primers that have not been incorporated into amplicons are removed in step (c) of the method of the first aspect using: (i) one or more endonuclease, wherein the endonuclease is selected from the group consisting of mung bean nuclease, nuclease P1, nuclease S1 and combinations thereof; or (ii) one or more exonuclease, wherein the exonuclease is selected from the group consisting of exonuclease I, exonuclease T, exonuclease VII and combinations thereof. In one example, the target capture primers that have not been incorporated into amplicons are removed in step (c) of the method of the first aspect using either exonuclease I, or exonuclease T, or a combination of both exonuclease I and exonuclease T. In one example, the target capture primers that have not been incorporated into amplicons are removed in step (c) of the method of the first aspect using exonuclease I. In one example, the target capture primers that have not been incorporated into amplicons are removed in step (c) of the method of the first aspect using exonuclease T. In one example, the target capture primers that have not been incorporated into amplicons are removed in step (c) of the method of the first aspect using a combination of both exonuclease I and exonuclease T. In one example, following the initial PCR step of the method of the present disclosure, comprising of a low number of PCR cycles (3 to 5 cycles) for target capture and limited amplification, excess target capture primers are removed by treatment with a blend of two exonucleases, exonuclease I and exonuclease T. In one example, the two exonucleases specifically digest unincorporated single-stranded primers in the 3′ to 5′ direction, while retaining double-stranded DNA products containing target regions of interest.

In one example, the method of the present disclosure comprises amplifying the plurality of amplicons in the purified mixture as defined in step (d) of the method of the first aspect by using universal indexed adapter primers to generate a sequencing library, wherein each amplicon of the sequencing library comprises two barcode sequences. In one example, the sequencing library generated comprises at least at least 1 amplicon, or at least 50 amplicons, or at least 100 amplicons, or at least 200 amplicons, or at least 300 amplicons, or at least 400 amplicons, or at least 500 amplicons, or at least 600 amplicons, or at least 700 amplicons, or at least 800 amplicons, or at least 900 amplicons, or at least 1000 amplicons, or at least 1100 amplicons, or at least 1200 amplicons, or at least 1300 amplicons, or at least 1400 amplicons, or at least 1500 amplicons, or at least 1600 amplicons, or at least 1700 amplicons, or at least 1800 amplicons, or at least 1900 amplicons, or at least 2000 amplicons, or at least 2100 amplicons, or at least 2200 amplicons, or at least 2300 amplicons, or at least 2400 amplicons, or at least 2500 amplicons, or at least 2600 amplicons, or at least 2700 amplicons, or at least 2800 amplicons, or at least 2900 amplicons, or at least 3000 amplicons. In one specific example, the sequencing library generated comprises at least 2600 amplicons. In one example, there is no upper limit for the number of amplicons in the generated sequencing library. In one example, only amplicons comprising barcode sequences and adapters on both their 5′ and 3′ ends are subjected to multiplex sequencing on a next-generation sequencing platform as defined in step (f) of the method of the first aspect. In one example, the sequencing library is subjected to an indexing PCR step where 1) further DNA amplification occurs to increase yields for sequencing and 2) predetermined index sequences are added to each sample to facilitate subsequent post-sequencing demultiplexing. In one example, final libraries with complete structures required for sequencing on the Illumina platform (FIG. 2B) are then pooled together for subsequent sequencing, after which the data is analysed using a bioinformatics pipeline.

In one example, the amplification is performed using KAPA Hifi HotStart ReadyMix (Roche), Phusion U Hot Start DNA Polymerase (Thermo Scientific), ZymoTaq DNA Polymerase (Zymo Research) and Q5U Hot Start High-Fidelity DNA Polymerase (NEB), etc.

In one example, each universal indexed adapter primer as disclosed in step (d) comprises an adapter sequence. In one example, the term “adapter sequence” refers to an oligonucleotide sequence bound to the 5′ and 3′ end of each DNA fragment in a sequencing library. The adapter sequences are complementary to the plurality of oligonucleotides present on the surface of the flow cells of the sequencing tools thereby allowing the DNA fragment to attach to the sequencing tool. In some examples, an adapter sequence allows for the sequencing of the oligonucleotide of interest. Sequencing platform specific adapter sequences are known in the art, and include, for example, the Illumina P5/P7 adapter sequences.

In one example, the universal indexed adapter primers as disclosed in step (d) of the method of the first aspect comprise:
a forward primer comprising the sequence of

(SEQ ID NO: 2)
AATGATACGGCGACCACCGAGATCTACACCTAGCGCTACACTCTTTCCC
TACACGACGCTCTTCCGATC*T;

and
a reverse primer comprising the sequence of

(SEQ ID NO: 3)
CAAGCAGAAGACGGCATACGAGATAACCGCGGGTGACTGGAGTTCAGAC
GTGTGCTCTTCCGATC*T,

wherein “*” represents a phosphorothioate bond, and wherein the underlined sequences are the barcode sequences.

In one example, the method of the present disclosure comprises purifying the sequencing library obtained from step (d) of the method of the first aspect. In one example, the purification of the plurality of sequencing library is performed using an agent such as paramagnetic beads. In one example, the paramagnetic beads are selected from the group consisting of AMPure XP beads, SPRI beads, and Dynabeads. In one specific example, the sequencing library is purified with two rounds of 0.8× volume AMPure XP beads to remove excess adapters and to size-select the final sequencing library.

In one example, the method of the present disclosure comprises subjecting the purified sequencing library from step (e) of the method of the first aspect to multiplex sequencing on a next-generation sequencing platform to obtain a plurality of sequencing reads. In one example, each final purified library was qualified using the High Sensitivity DNA Screentape (Agilent) and quantified using KAPA Library Quantification Kit (Roche) before being sequenced on an NGS platform. In one example, suitable kits for qualifying and quantifying the final purified library include NEBNext Library Quant Kit (New England Biolabs), Collibri Library Quantification Kit (Thermo Fisher) and ProNex NGS Library Quant Kit (Promega). In some examples, the NGS platform is NextSeq 550, NextSeq 2000, NovaSeq 6000, BGI MGISEQ-2000, DNBSEQ-G400 or DNBSEQ-T7.

In one example, the method of the present disclosure further comprises mapping the plurality of sequencing reads obtained from step (f) of the method of first aspect to a first reference genome. In one example, the term “mapping” refers to the process of aligning sequencing reads to a reference genome. In one example, the term “reference genome” refers to DNA sequences known in the art that may be obtainable from public databases.

In one example, the method of the present disclosure further comprises grouping sequencing reads where the barcode sequences of the sequencing reads are identical into a consensus cluster. The term “consensus cluster” refers to a group of consensus sequence reads wherein at least one of the two barcode sequences of the consensus sequence reads are identical. In one example, the term “consensus cluster” refers to a group of consensus sequence reads wherein one of the two the barcode sequences of the consensus sequence reads are identical. In one example, the term “consensus cluster” refers to a group of consensus sequence reads wherein the two barcode sequences of the consensus sequence reads are identical.

In one example, each consensus cluster as defined in step (h) of the method of the first aspect comprises at least 1 family member. In one example, each consensus cluster as defined in step (h) of the method of the first aspect comprises at least 2 family members. In one example, each consensus cluster as defined in step (h) of the method of the first aspect comprises at least 3 family members. In one example, each consensus cluster as defined in step (h) of the method of the first aspect comprises at least 4 family members. In one example, each consensus cluster as defined in step (h) of the method of the first aspect comprises at least 5 family members. In one example, each consensus cluster as defined in step (h) of the method of the first aspect comprises at least 6 family members. In one example, each consensus cluster as defined in step (h) of the method of the first aspect comprises at least 7 family members. In one example, each consensus cluster as defined in step (h) of the method of the first aspect comprises at least 8 family members. In one example, each consensus cluster as defined in step (h) of the method of the first aspect comprises at least 9 family members. In one example, each consensus cluster as defined in step (h) of the method of the first aspect comprises at least 10 family members. In one example, there is no upper limit for the number of family members in each consensus cluster as defined in step (h) of the method of the first aspect. In one example, the method of the present disclosure further comprises performing a second sequence alignment of each consensus cluster obtained from step (h) with all consensus clusters having at least one overlapping barcode sequence. In one example, the term “overlapping barcode sequence” refers to a common barcode sequence between consensus sequencing reads with at least one identical barcode sequence. In one example, prior to performing step (i) of the method of the first aspect, consensus clusters with fewer than 1 to 5 members are considered unreliable and removed prior to downstream analyses. In one example, prior to performing step (i) of the method of the first aspect, consensus clusters with fewer than 5 family members are removed from downstream analysis. In one example, prior to performing step (i) of the method of the first aspect, consensus clusters with fewer than 4 family members are removed from downstream analysis. In one example, prior to performing step (i) of the method of the first aspect, consensus clusters with fewer than 3 family members are removed from downstream analysis. In one example, prior to performing step (i) of the method of the first aspect, consensus clusters with fewer than 2 family members are removed from downstream analysis.

In one example, prior to performing step (g) of the method of the first aspect (I) bases having poor quality scores are removed, and (II) universal indexed adapter primers and barcode sequences are trimmed. In one example, the bases having poor quality scores are removed by replacing the bases with “N”. In one example, the universal indexed adapter primers and barcode sequences are trimmed using bioinformatic tools. In one example, the universal indexed adapter primers and barcode sequences are trimmed using Cutadapt.

In one example, the mapping in steps (g) and (j) and the sequence alignment in step (i) of the method of the first aspect are performed using bioinformatic algorithms. In one example, the mapping in steps (g) and (j) of the method of the first aspect is performed using bioinformatic algorithms. In one example, the sequence alignment in step (i) of the method of the first aspect is performed using bioinformatic algorithms. In one example, the mapping in steps (g) and U) are performed using bwa-mem. It is known in the art that Burrows-Wheeler Alignment tool (BWA) is a software package for mapping low-divergent sequences against a large reference genome, such as the human genome. In one example, the sequence alignment in step (i) is performed using MAFFT.

In one example, the method of the present disclosure further comprises mapping each of the consensus sequencing read from step (i) of the method of the first aspect with a second reference genome.

In one example, the first reference genome as defined in step (g) of the method of the first aspect and the second reference genome as defined in step U) of the method of the first aspect comprise identical gene sequences.

In one example, the method of the present disclosure further comprises identifying the differences between the sequencing read and the reference genome from step U) of the method of the first aspect to thereby determine the presence of genomic alteration in the nucleic acid present in the biological sample.

In one example, the biological sample is obtained from a subject having and/or suspected of having a disease. In one example, the biological sample is obtained from a subject having a disease. In one example, the biological sample is obtained from a subject suspected of having a disease. In one example, the disease is cancer. In one example, the disease is an infectious disease.

In one example, the cancer is selected from the group consisting of leukemia, lung cancer, colorectal cancer, breast cancer, pancreatic cancer, prostate cancer, nasopharyngeal cancer, liver cancer, cholangiocarcinoma, esophageal cancer, urothelial cancer, and gastrointestinal cancer.

In one example, the infectious disease is viral infection or bacterial infection.

In a second aspect, the present disclosure refers to a kit for detecting genomic alterations within a biological sample according to the method disclosed herein, comprising a plurality of target capture primer pairs specific to a plurality of target genes that are capable of undergoing genomic alteration as defined in step (b) of the method of the first aspect, and instructions for use in the method disclosed herein.

In one example, the kit further comprises: a buffer for performing a plurality of multiplexed PCR reactions, a DNA polymerase, a plurality of deoxynucleoside triphosphates (dNTPs), and one or more nucleases capable of removing unincorporated target capture primers. In some examples, the reagents provided in the kit as described herein may be provided in separate containers comprising the components independently distributed in one or more containers. As the method as described herein relates to sequencing (such as high-throughput sequencing), further components required in sequencing process could be easily determined by the person skilled in the art.

As used in this application, the singular form “a,” “an,” and “the” include plural references unless the context clearly dictates otherwise. For example, the term “a primer” includes a plurality of primers, including mixtures and combinations thereof.

As used herein, the terms “increase” and “decrease” refer to the relative alteration of a chosen trait or characteristic in a subset of a population in comparison to the same trait or characteristic as present in the whole population. An increase thus indicates a change on a positive scale, whereas a decrease indicates a change on a negative scale. The term “change”, as used herein, also refers to the difference between a chosen trait or characteristic of an isolated population subset in comparison to the same trait or characteristic in the population as a whole. However, this term is without valuation of the difference seen.

As used herein, the term “about” in the context of concentration of a substance, size of a substance, length of time, or other stated values means +/−5% of the stated value, or +/−4% of the stated value, or +/−3% of the stated value, or +/−2% of the stated value, or +/−1% of the stated value, or +/−0.5% of the stated value.

Throughout this disclosure, certain embodiments may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The present disclosure illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the disclosure claimed. Thus, it should be understood that although the present disclosure has been specifically disclosed by preferred embodiments and optional features, modification and variation of the present disclosure embodied therein herein disclosed may be resorted to by those skilled in the art, and that such modifications and variations are considered to be within the scope of this present disclosure.

The disclosure has been described broadly and generically herein. Each of the narrower species and subgeneric groupings falling within the generic disclosure also form part of the present disclosure. This includes the generic description of the present disclosure with a proviso or negative limitation removing any subject matter from the genus, regardless of whether or not the excised material is specifically recited herein.

Other embodiments are within the following claims and non-limiting examples.

### EXAMPLES

### Materials

Exemplary Molecular Tag Complex or Primers when Target is EGFR_Exon19

An example of a “primer” when the target sequence is EGFR_exon19 (an example of forward target capture primer, illustrated in FIG. 2A) is as follows:

(SEQ ID NO: 4)
ACACGACGCTCTTCCGATCTNNNNNNNNNNTGCCAGTTAACGTCTTCCT
TCTC,

wherein the bases in italic and underline are an example of adapter sequence, the bases in bold represent the barcode sequence and the bases in underline is an example of target specific sequence.

An example of subsequent primers for the “completion of amplicon” (an example of a reverse target capture primer, illustrated also in FIG. 2A) is as follows:

(SEQ ID NO: 5)
GACGTGTGCTCTTCCGATCTNNNNNNNNNNCCACACAGCAAAGCAGAAA
CTC,

wherein the bases in italic and underline are an example of adapter sequence, the bases in bold represent the barcode sequence and the bases in underline is an example of target specific sequence.

### Expected Amplicon (Only Target-Specific Region)

>chr7:55242380+55242537 158 bp

(SEQ ID NO: 6)
TGCCAGTTAACGTCTTCCTTCTCtctctgtcatagggactctggatccc
agaaggtgagaaagttaaaattcccgtcgctatcaaggaattaagagaa
gcaacatctccgaaagccaacaaggaaatcctcgatgtGAGTTTCTGCT
TTGCTGTGTGG

Product after Amplicon Completion (in Two Steps) (Only One Strand of the Double Stranded Product is Shown.):

(SEQ ID NO: 7)
ACACGACGCTCTTCCGATCTNNNNNNNNNNTGCCAGTTAACGTCTTCCT
TCTCtctctgtcatagggactctggatcccagaaggtgagaaagttaaa
attcccgtcgctatcaaggaattaagagaagcaacatctccgaaagcca
acaaggaaatcctcgatgtGAGTTTCTGCTTTGCTGTGTGGNNNNNNNN
NNAGATCGGAAGAGCACACGTC,

where the bases in underline is target nucleic acid.

### Final Product, Illustrated Also in FIG. 2B (Suitable for Sequencing on Illumina)

(SEQI D NO: 8)
AATGATACGGCGACCACCGAGATCTACACCTAGCGCTACACTCTTTCCC
TACACGACGCTCTTCCGATCTNNNNNNNNNNTGCCAGTTAACGTCTTCC
TTCTCtctctgtcatagggactctggatcccagaaggtgagaaagttaa
aattcccgtcgctatcaaggaattaagagaagcaacatctccgaaagcc
aacaaggaaatcctcgatgtGAGTTTCTGCTTTGCTGTGTGGNNNNNNN
NNNAGATCGGAAGAGCACACGTCTGAACTCCAGTCACCCGCGGTTATCT
CGTATGCCGTCTTCTGCTTG,

where the bases in underline is target nucleic acid.

### Methods

### Sample Collection and Processing

Blood collected in Cell-free DNA BCT (Streck) was shipped at ambient temperature before plasma separation. Plasma was prepared using a two-step centrifugation process: the first centrifugation was done at 1600×g for 10 min at 4° C. to separate plasma. The plasma layer was then transferred to a separate tube and centrifuged at 16,000×g for 10 min at 4° C. to further remove cellular contaminants, and immediately processed for nucleic acid extraction or stored at −80° C. until used for extraction. If frozen, the plasma was fully thawed at room temperature before extraction.

Cell-free total nucleic acids were extracted from 3 to 5 mL of plasma using the QIAamp Circulating Nucleic Acid kit (Qiagen). cfDNA was quantified using the Qubit 1× dsDNA High Sensitivity kit (Thermo Fisher Scientific).

### Preparation of Sequencing Library

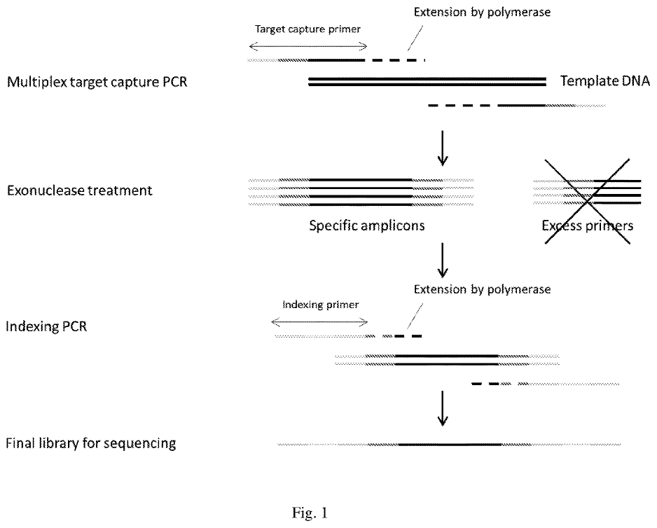

The generation of a sequencing library is achieved in three steps as illustrated in FIG. 1:
1. Barcode sequence assignment and amplicon generation (Multiplex target capture PCR)
2. Removal of excess target capture primers (Exonuclease treatment)
3. Final library amplification (Indexing PCR)

### Barcode Sequence Assignment and Amplicon Generation

In this first step, target DNA molecules are captured with a pair of primers per target. Each target capture primer is composed of three parts—the target-specific sequence, a 10-base pair random nucleotide sequence (NNNNNNNNNN) (SEQ ID NO: 1) upstream of the target-specific sequence, and an adapter-specific sequence (FIG. 2A). The target-specific sequence achieves target capture, the 10-base pair random nucleotide constitutes the “unique barcode sequence”, and the adapter-specific sequence serves as the primer landing site for the final library amplification primers. The combination of the target-specific sequence and the 10-base pair unique barcode sequence for both forward and reverse primers were used to trace and define a unique original parental DNA molecule.

cfDNA was used as a template in a highly multiplexed PCR reaction for target capture using the Platinum™ SuperFi II DNA Polymerase (Thermo Fisher Scientific). Briefly, in a 50 μl PCR reaction, cfDNA was mixed with target capture primers at a final concentration of 10 to 100 nM (each primer), 10 μl of 5× SuperFi II Buffer, 10 nM dNTPs, and 2 μl Platinum SuperFi II DNA Polymerase, and subjected to the following thermocycling conditions: initial denaturation at 98° C. for 1 minute; followed by 3 to 5 cycles of denaturation at 98° C. for 10 seconds, annealing at 58° C. for 6 minutes, extension at 72° C. for 5 minutes; and lastly a final extension at 72° C. for 5 minutes.

### Removal of Excess Target Capture Primers

The PCR product underwent exonuclease treatment by adding 6.1 μl 10× NEBuffer r3.1 (NEB), 2.5 μl thermolabile exonuclease I (NEB) and 2.5 μl exonuclease T (NEB), followed by an incubation at 37° C. for 10 min. The exonuclease-treated product was then subjected to clean-up using 1.5× volume of AMPure XP beads (Beckman Coulter), and eluted in 23 μl of Buffer EB (Qiagen).

### Final Library Amplification

Purified products were then amplified with universal indexed adapter primers (to introduce sample indexes and Illumina sequencing adapters) in a 50 μl reaction with 2 μM (final concentration) primers using KAPA HiFi HotStart ReadyMix (Roche). The PCR was carried out with the following thermocycling profile: initial denaturation at 98° C. for 45 seconds; followed by 14 to 16 cycles of denaturation at 98° C. for 15 seconds, annealing at 60° C. for 30 seconds, extension at 72° C. for 30 seconds; and lastly a final extension at 72° C. for 1 minute. The amplified library was purified with two rounds of 0.8× volume AMPure XP beads to remove excess adapters and to size-select the final sequencing library. Each final purified library (FIG. 2B) was qualified using the High Sensitivity DNA Screentape (Agilent) and quantified using KAPA Library Quantification Kit (Roche) before being sequenced on a NextSeq 550 system (Illumina).

### Data Analysis

Binary base call sequencing files were first demultiplexed and converted to FASTQ files, which are processed using a custom pipeline. First, bases with poor quality scores were filtered. Next, read 1 and corresponding read 2 FASTQ files were searched for expected forward and reverse primer sequences respectively, based on an input file containing named primer sequences of all amplicons within the panel. Primer sequences and upstream barcode sequences were trimmed using Cutadapt and the trimmed sequences were mapped to the reference genome using bwa-mem. Reads were annotated with their corresponding primer names. The primer name assigned to read 1 may not always match that of read 2 due to overlapping amplicons or non-specific binding. An “amplicon_name” was assigned to each read pair by concatenating the matching primer name of reads 1 and 2 (F_name;R_name). Barcode sequences from both reads 1 and 2 were also concatenated and assigned separately to each paired read (F_barcode;R_barcode).

Subgraph consensus clustering of barcode sequences was performed by considering each amplicon_name as a network. Each read assigned the same amplicon_name was represented within the amplicon_name network as a subgraph of two connected nodes of identity F_barcode and R_barcode. Every subsequent read was added to the network either as a disconnected subgraph or joined to an existing subgraph via a common barcode (either F_barcode or R_barcode), until no more reads were left. Each consensus cluster was a disconnected subgraph within the network and was represented by the amplicon_name appended with a number (amplicon_name_n), representing the number of disconnected subgraphs for each amplicon. Consensus clusters with fewer than 1 to 5 members were considered unreliable and removed prior to downstream analyses.

Consensus calling was done for each consensus cluster, first via global alignment of all consensus family members using MAFFT. The consensus base in each aligned position was called by determining the majority representative base, the percentage of which was no less than an automatically determined threshold, which is a function of the total number of reads within the consensus cluster. If no representative base can be called, the position was assigned N, as opposed to one of A, C, T, G. A new quality score was assigned to each position, which was either 90th percentile of all the quality values from the representative base type in that position if a consensus base was found, or 10th percentile of all quality values in that position if no consensus base was found. The consensus reads are written to new consensus FASTQ files, which were then mapped to the reference genome with local realignment to improve mapping. Consensus read depth was calculated from the mapped BAM file as the unique number of consensus clusters mapped to each target region specified in the panel. Variant calling was performed on consensus BAM files using a custom variant caller.

### Results

### Validation of the Exonuclease Treatment Step

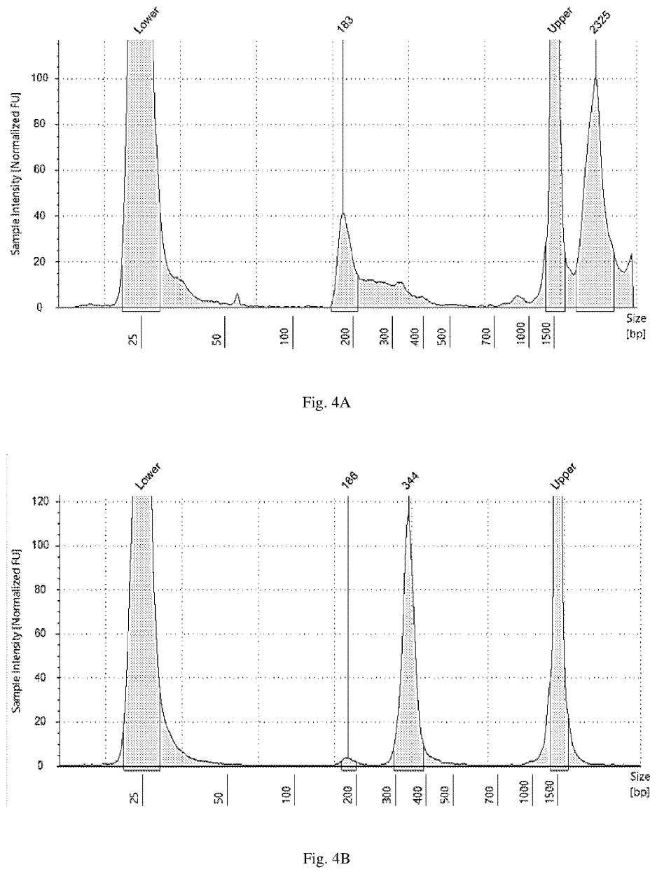

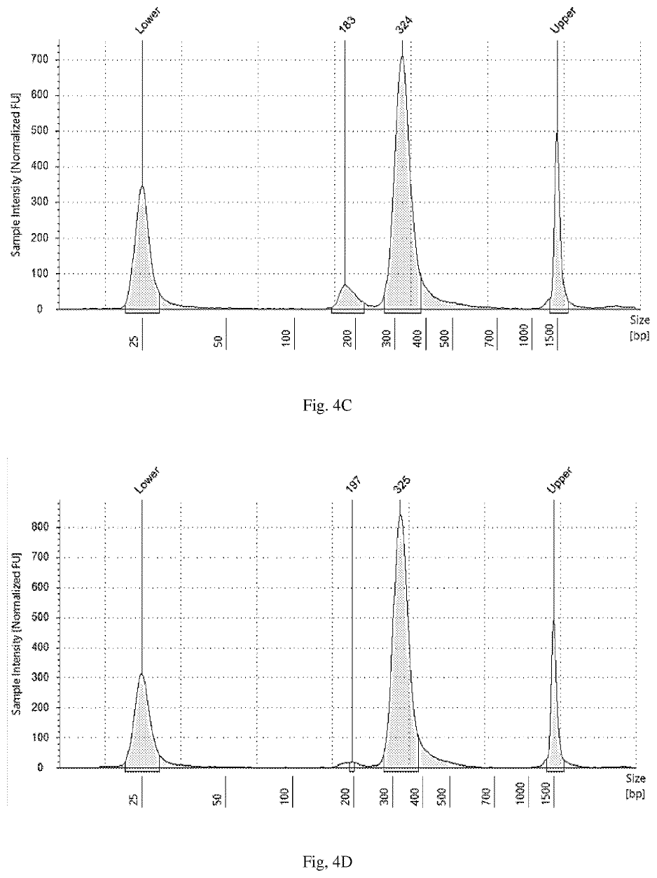

Following the initial PCR, comprising of a low number of PCR cycles (3-5 cycles) for target capture and limited amplification, excess target capture primers were removed by treatment with a blend of two exonucleases, exonuclease I and exonuclease T. The two exonucleases specifically digested unincorporated single-stranded primers in the 3′ to 5′ direction, while retaining double-stranded DNA products containing target regions of interest. As demonstrated in FIGS. 4A, 4B, 4C and 4D and Table 1 (a comparison of library profiles without exonuclease and with exonuclease treatment for an increasing number of amplicons in exemplary panels), the method described herein can generate libraries from primers representing greater than 2600 amplicons (FIG. 4D), while retaining a high proportion of specific products. Table 1 shows the sequencing QC metrics of representative libraries generated from a workflow incorporating exonuclease treatment for an increasing number of amplicons in exemplary panels. As shown in Table 1, high percentage reads on target (88.17%), high uniformity (88.99%) and high average consensus depth were achieved even with a large panel size of 2624 amplicons.

TABLE 1

| | Panel Size (No. of amplicons) | | |
|---|---|---|---|
| QC metric | 334 | 1167 | 2624 |
| % Reads on target | 85.69% | 76.88% | 88.17% |
| Uniformity (% ≥0.2x | 96.97% | 86.28% | 88.99% |
| consensus depth) | | | |
| Average consensus | 952.96 | 801.82 | 844.69 |
| depth per ng input | | | |

### Comparison of the Subgraph Consensus Clustering Analysis to Conventional Standard Barcode Clustering Analysis

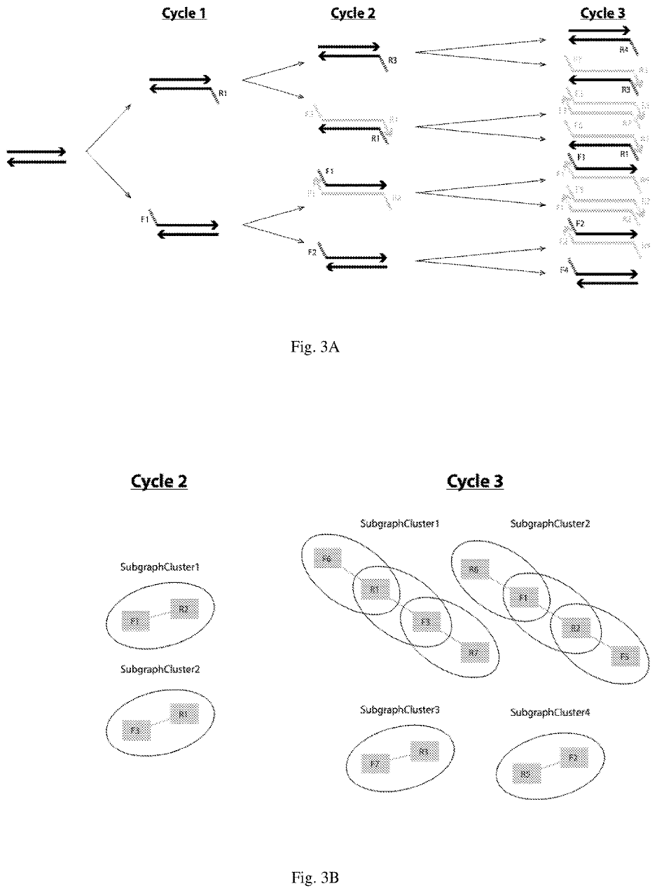

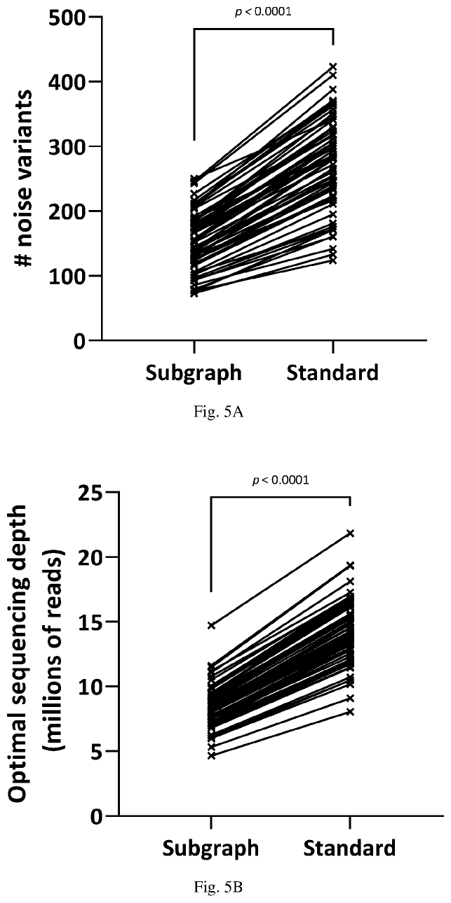

To confirm the error reduction rate and cost-efficiency of the subgraph consensus clustering analysis workflow, 65 cfDNA samples (2.5 ng) were sequenced using an NGS panel based the method disclosed herein, and both the subgraph consensus clustering and the conventional standard barcode clustering analysis workflows were applied separately to the FASTQ files. The conventional barcode clustering analysis trivially considers each unique F and R barcode combination as representing a unique DNA template molecule, and does not account for the barcode duplication that is likely to happen from cycle 3 onwards of PCR (FIG. 3A). The mean number of noise variants in the subgraph consensus clustering workflow was 155, compared to 273 using a standard barcode clustering analysis workflow, representing a mean 43.6% reduction in error rate (paired t-test p-value<0.0001) (FIG. 5A). In pair-wise comparison (across 65 cfDNA samples) of the optimal sequencing depth required to obtain an average consensus family size of 10 (based on linear extrapolation), the mean number of reads required was 8.5 million for the subgraph consensus clustering workflow, compared to 14.2 million for the standard analysis workflow, for a panel size of 134 kb. These results demonstrate a 40.4% reduction in required reads (paired t-test p-value<0.0001) (FIG. 5B). These results show that the subgraph consensus clustering workflow significantly reduced error rates and the required sequencing depth, which translate to more accurate detection (with reduced error rates) and higher sequencing cost-efficiency compared to the conventional standard barcode clustering analysis workflow.

### Validation of the Sensitivity of the Consensus Clustering Workflow

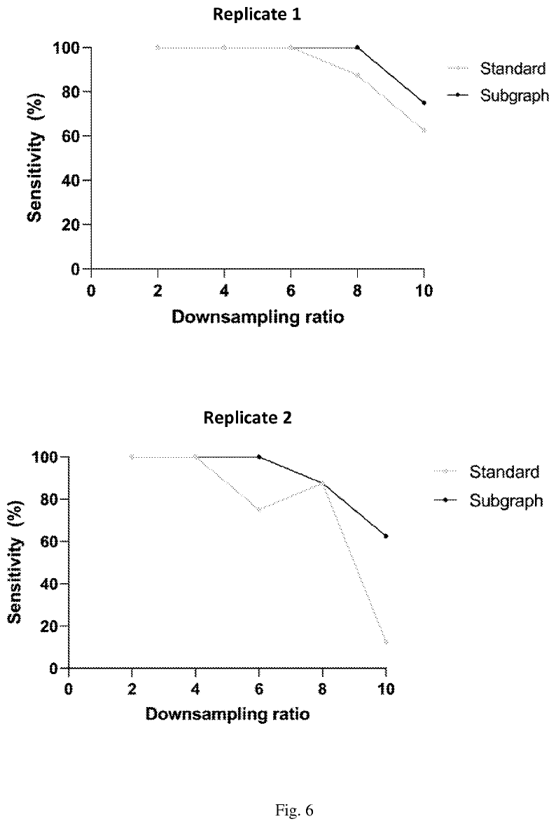

To demonstrate the high sensitivity of the disclosure for detection of low variant allele frequencies (VAF), a multiplex panel designed with features disclosed herein was used to interrogate the Horizon Discovery™ Multiplex I cfDNA Reference Standard (HD779) containing eight alterations previously characterized to be at 0.1% to 0.13% VAF. Results from 7 replicates using a limiting amount of input DNA (30 ng) demonstrated a sensitivity of 89.3% (n=56 variants), with a high degree of accuracy of VAF calls (Table 2). To confirm that high sensitivity of the method is retained at reduced reads, generated FASTQ files from two replicates were downsampled 2× to 10× to simulate a 50% to 90% reduction in sequencing reads and analysed both using a standard barcode clustering analysis workflow and the subgraph consensus clustering workflow. Application of both analysis workflows on the same FASTQ files revealed that the subgraph consensus clustering method possessed equivalent or superior sensitivity at each sequencing read depth simulated, with superior sensitivity apparent when the sequencing read depth was reduced beyond 80% of the original read depth (FIG. 6). This is explained by the increase in numbers of true family members belonging to a given consensus family, that happens due to the generation of subgraphs sharing either F_barcode or R_barcode (accurate inclusion of more members of consensus family in a consensus cluster) which in turn drives up the accuracy of consensus calls. This effect becomes particularly apparent when the total number of reads is limiting, and standard clustering analysis results in sparsely populated barcode families which are not optimal for accurate consensus calling, impacting sensitivity of detection at low VAFs.

TABLE 2

| | Expected | Observed VAF (%) in each replicate (Rep) | | | | | | |
|---|---|---|---|---|---|---|---|---|
| Variant | VAF (%) | Rep 1 | Rep 2 | Rep 3 | Rep 4 | Rep 5 | Rep 6 | Rep 7 |
| EGFR L858R | 0.10 | 0.09 | 0.06 | 0.18 | 0.13 | 0.12 | 0.04 | 0.09 |
| EGFR E746_A750del | 0.10 | 0.25 | 0.09 | 0.10 | 0.11 | 0.06 | 0.16 | 0.19 |
| EGFR T790M | 0.10 | 0.10 | 0.12 | 0.17 | 0.13 | ND | 0.06 | 0.04 |
| EGFR A767_V769dup | 0.10 | 0.09 | 0.15 | 0.05 | ND | 0.15 | ND | ND |
| KRAS G12D | 0.13 | 0.14 | 0.11 | 0.08 | 0.09 | 0.33 | ND | ND |
| NRAS Q61K | 0.13 | 0.15 | 0.18 | 0.06 | 0.14 | 0.13 | 0.13 | 0.15 |
| NRAS A59T | 0.13 | 0.27 | 0.09 | 0.23 | 0.07 | 0.10 | 0.06 | 0.06 |
| PIK3CA E545K | 0.13 | 0.22 | 0.16 | 0.22 | 0.04 | 0.16 | 0.11 | 0.10 |

Detection of Genomic Alteration in cfDNA

To demonstrate the application of the detection method disclosed herein in cfDNA sample, five plasma cfDNA samples were sequenced using the workflow described in the present disclosure with dual barcodes, and results were compared against variant calls made using a single barcode sequence method. To confirm the sensitivity of the method of the present disclosure at reduced sequencing reads, libraries were sequenced at a higher sequencing depth and analysed first using a standard barcode analysis workflow. Subsequently, generated FASTQ files were downsampled, reducing the sequencing read depth by 13% to 42%, and the downsampled files were analysed using the subgraph consensus clustering workflow. In all five cfDNA samples, all previously detected variants, ranging from 0.17% to 7.59% VAF, were identified using this method, both using the standard barcode clustering workflow as well as the subgraph consensus clustering workflow at reduced read depths (Table 3). In one sample, an EGFR exon 20 insertion not previously identified was also detected with high confidence using the method disclosed herein. Collectively, these results demonstrate the high sensitivity of detection of the method of the present disclosure, particularly at reduced sequencing read depths.

TABLE 3

| | | | Current method | | |
|---|---|---|---|---|---|
| | | | VAF (%) | VAF (%) from | % reduction |
| | | | from | subgraph | in sequencing |
| | Single molecular barcode method | | standard | consensus | read depth |
| | | VAF | barcode | clustering with | after |
| Sample | Variant | (%) | analysis | reduced reads | downsampling |
| 1 | EGFR E746_A750del | 7.59 | 11.24 | 11.46 | 21.6 |
| | TP53 P152L | 5.79 | 5.7 | 5.57 | |
| 2 | EGFR L858R | 0.57 | 0.61 | 0.62 | 31.1 |
| | TP53 C124Afs * 46 | 0.37 | 0.23 | 0.14 | |
| 3 | ERBB2 Y772_A775dup | 5.54 | 2.68 | 2.6 | 14.4 |
| | TP53 c.559 + 1G > A | 3.95 | 4.52 | 4.05 | |
| 4 | EGFR N771_H773dup | 0.82 | 1.17 | 1 | 13.4 |
| | TP53 Q317Sfs * 28 | 0.17 | 0.29 | 0.27 | |
| 5 | EGFR | ND | 0.16 | 0.16 | 42.1 |
| | H773_V774insGHPH | | | | |

### DISCUSSION

The present disclosure describes experimental and data analysis workflows which enable the highly multiplexed detection of rare genetic alterations with high sensitivity, high sequencing cost-efficiency, and reduced error rates using a small nucleic acid sample input amount. The incorporation of barcode sequences in both the forward and reverse primers during initial DNA target capture allows information to be captured from both strands of DNA in the double-stranded DNA input, an approach that has not previously been adopted in amplicon-based target capture. The overall doubling of DNA target capture as described herein improves the sensitivity of conventional methods known in the art for which target capture is only performed on one DNA strand of the double-stranded DNA source material

The incorporation of unique barcode sequences during DNA target capture in next-generation sequencing (NGS)-based methods enables an approximate 100-fold error correction as compared to standard Illumina NGS. The traceable capture of both strands of double-stranded cfDNA using barcode sequences, in the manner described above, would in theory increase the confidence of detection of low-level variants, by increasing the amount of sequence information captured. Errors potentially introduced in one strand and not the other during the earliest steps in target capture (which can continue to be propagated), could be further suppressed by taking into consideration sequence information from both captured strands. In the present disclosure, barcode sequences are incorporated in both the forward and reverse primers during PCR-based DNA target capture. Both forward and reverse primers are in the same PCR reaction allowing the tagging and capture of both DNA strands of a double-stranded DNA molecule and the formation of dually-barcoded amplicons. The overall doubling of DNA target capture is therefore expected to improve the sensitivity of the method in detecting genomic alterations, including but not limited to single-nucleotide variations, insertions/deletions, genomic copy number alterations, deletions of homopolymeric regions, total mutation (or variant) load, detection of microbial DNA sequences and detection of polymorphisms or single-nucleotide variations in microbial DNA sequences.

The traceable capture of both strands of double-stranded cfDNA using barcode sequences, would in theory increase the confidence of detection of low-level variants, by increasing the amount of sequence information captured. Errors potentially introduced in one strand and not the other during the earliest steps in target capture (which can continue to be propagated), could be further suppressed by taking into consideration sequence information from both captured strands. In the present disclosure, barcode sequences are incorporated in both the forward and reverse primers during PCR-based DNA target capture. Both forward and reverse primers are in the same PCR reaction allowing the tagging and capture of both DNA strands of a double-stranded DNA molecule and the formation of dually-barcoded amplicons. The overall doubling of DNA target capture is therefore expected to improve the sensitivity of the method in detecting genomic alterations, including but not limited to single-nucleotide variations, insertions/deletions, genomic copy number alterations, deletions of homopolymeric regions, total mutation (or variant) load, detection of microbial DNA sequences and detection of polymorphisms or single-nucleotide variations in microbial DNA sequences.

The inclusion of the exonuclease treatment step prevents the carryover of target capture primers to the subsequent indexing PCR where further amplification occurs, thus minimising the formation of undesirable non-specific products, such as primer-dimers, in the final library. The addition of the exonuclease treatment step significantly expands the number of primers that can be accommodated in the initial target capture PCR without compromising the quality of the final library, which allows the breadth of the panel to be scaled up to include more amplicons targeting additional regions of interest, by economizing sequencing resources for specific target entities. Following exonuclease treatment, the library is subjected to an indexing PCR step where 1) further DNA amplification occurs to increase yields for sequencing and 2) predetermined index sequences are added to each sample to facilitate subsequent post-sequencing demultiplexing. Final libraries with complete structures required for sequencing on the Illumina platform (FIG. 2B) are then pooled together for sequencing, after which the data is analysed using a bioinformatics pipeline.

To account for the inclusion of barcode sequences to capture both strands of DNA, the method of the present disclosure incorporates a modification in the consensus sequence read generation from constituent barcoded reads in the sequencing data analysis. The additional information provided by two barcode sequences (in comparison to a single barcode sequence) is also used to identify amplicons that arise due to PCR-mediated barcode duplication, and are not in fact representative of true unique DNA template molecules. Such amplicons belong to the same consensus sequence family (representing same original double stranded DNA template) but possess unique combinations of barcode sequences (therefore appearing unique in simplistic analysis (FIG. 3A). This contrasts with the desired capture and representation of unique original DNA molecules present in the original sample. The advantages of the modified consensus clustering workflow, termed subgraph consensus clustering, are two-fold. First, the method of the present disclosure minimises inflation of consensus coverage arising due to barcode duplication/resampling (FIG. 3B), enabling sequencing at much lower read depths to achieve similar sensitivities and improving the cost-effectiveness of the overall method. Second, by capturing both strands of DNA, followed by more accurately assigning amplicons to the correct consensus family, the error detection (and suppression) rate of the method disclosed herein is enhanced.

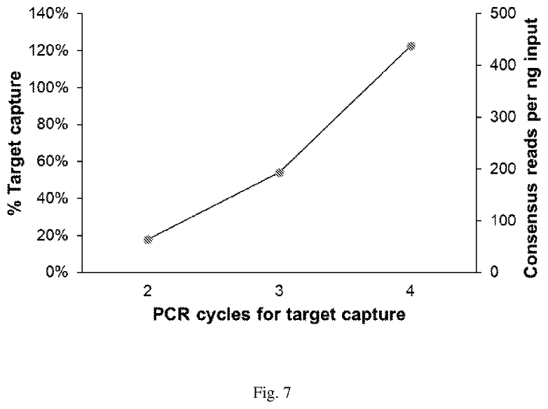

The initial target capture step of the method disclosed herein involves limited amplification with a minimal number of PCR cycles (3 to 5 cycles) to maximise the chances of recovering representative molecules from the limited amount of DNA source material. In an ideal setting, each unique combination of forward and reverse barcode sequences will represent information from a single unique template strand, and both strands of dsDNA would be captured and converted to sequenceable or universally amplifiable products in exactly two cycles. However, PCR is a less than 100% efficient process per cycle, particularly at limiting template copy numbers, and in order to maximise the barcode capture of available DNA templates, in practice more than two cycles is required (FIG. 7). This also means that in the initial target capture step, though cycling is limited, some barcoded DNA strands will possibly undergo re-barcoding (or barcode duplication/resampling), resulting in excess duplication of original template molecule and thus inefficient sequencing. The subgraph consensus clustering of barcode sequences described herein addresses this issue by allowing related barcode families to be deduplicated, which means sequencing can be performed at lower read depths while retaining a similarly high level of sensitivity, thus improving the cost-effectiveness of the overall method. Furthermore, error rates will be reduced and quantification will be improved with the increase in accuracy of assigning amplicons to the correct consensus family.

While amplicon-based sequencing offers technical advantages over hybridization capture-based approaches in terms of a faster workflow and better sensitivity at low frequencies, which is of particular importance in liquid biopsies, amplicon-based methods are generally less amenable towards accommodating increases in panel breadth. As such, there is an urgent need for innovative approaches to increase the scalability of amplicon-based sequencing. Since input material is often limited in liquid biopsies due to biology, increasing the breadth of panel coverage would require the inclusion of more target capture primers within a single PCR reaction. To increase the scalability of the method, an exonuclease digestion step has been included after the initial target capture PCR. The PCR product is treated with a blend of 2 different exonucleases, which remove excess, unincorporated single-stranded primers while retaining double-stranded targets of interest. The removal of excess unincorporated primers will minimize the formation of undesirable non-specific products, such as primer-dimers, while maximizing the yield of desired targets in downstream steps involving further PCR amplification. This allows the panel of target capture primers to be scaled up to a more comprehensive level, without compromising performance. FIGS. 4A, 4B, 4C and 4D and Table 1 demonstrate the utility of our method in generating libraries with primers representing greater than 2600 amplicons, while retaining a high proportion of specific products.

The present disclosure describes for the first time:
1. The inclusion of barcode sequences in target capture primers which are specifically designed to capture information from both the plus and minus strands of DNA in target regions of interest, which allows for highly sensitive and accurate detection and quantification of genetic alterations.
2. The ability to identify and deduplicate related barcode sequence families via subgraph consensus clustering, enabling sequencing at much lower read depths while retaining similar sensitivities and improving cost-effectiveness.
3. Subgraph consensus clustering described herein increases the accuracy of assigning amplicons to the correct consensus family, which will reduce error rates and increase quantification accuracy.
4. The addition of an exonuclease treatment step to remove excess unincorporated primers, which have detrimental effects on downstream library generation, allows primers targeting additional regions to be multiplexed in the initial PCR without affecting the quality of the final library, thus drastically improving the scalability of the method in terms of increasing panel breadth.

The most important features of the method disclosed herein include:
1. Unique design of primers and methodological features which allow simultaneous detection of multiple classes of genetic alterations from same (often limiting) DNA source.
2. Unique design of bioinformatics pipeline that exploits the technology features to accurately capture unique target molecules from multiple originating sources, including from a mixture of sources.
3. The expandability of the target regions, with limited optimization needed by inclusion of appropriate primers.
4. The flexibility of incorporating new targets as and when those of clinical relevance are incorporated into clinical practice.

The method of the present disclosure has the following advantages:
1. The method of the present disclosure combines the use of uniquely designed primers and a modified consensus clustering workflow, which allows for highly multiplexed detection of rare genetic alterations with high sensitivity, high sequencing cost-efficiency, and reduced error rates using a small nucleic acid sample input amount.
2. The method of the present disclosure involves the inclusion of two molecular barcodes in target capture primers which are specifically designed to capture information from both the plus and minus strands of DNA in target regions of interest, increasing the sensitivity and accuracy of the detection and quantification of rare genetic alterations.
3. The addition of an exonuclease treatment step to remove excess unincorporated primers allows primers targeting additional regions to be multiplexed in the initial PCR, drastically improving the scalability of the method in terms of increasing panel breadth, thus improving the cost-effectiveness of the detection method.
4. The method of the present disclosures utilizes the subgraph consensus clustering data analysis workflow, increasing the accuracy of assigning amplicons to the correct consensus family, which ultimately reduced error rates and increase quantification accuracy of the detection method.

### SEQUENCE LISTING

| SEQ ID | | |
|---|---|---|
| NO. | Sequence Name | Sequence |
| 1 | Barcode sequence | NNNNNNNNNN |
| 2 | Universal indexed adapter | AATGATACGGCGACCACCGAGATCTAC |
| | forward primer | ACCTAGCGCTACACTCTTTCCCTACACG |
| | | ACGCTCTTCCGATC*T |
| 3 | Universal indexed adapter | CAAGCAGAAGACGGCATACGAGATAAC |
| | reverse primer | CGCGGGTGACTGGAGTTCAGACGTGTG |
| | | CTCTTCCGATC*T |
| 4 | Exemplary forward target | ACACGACGCTCTTCCGATCTNNNNNNNN |
| | capture primer if the target | NNTGCCAGTTAACGTCTTCCTTCTC |
| | sequence is EGFR_exon19 | |
| 5 | Exemplary reverse target | GACGTGTGCTCTTCCGATCTNNNNNNNN |
| | capture primer if the target | NNCCACACAGCAAAGCAGAAACTC |
| | sequence is EGFR_exon19 | |
| 6 | Exemplary expected | TGCCAGTTAACGTCTTCCTTCTCtctctgtcata |
| | amplicon (only target- | Gggactctggatcccagaaggtgagaaagttaaaattcccgtcgc |
| | specific region) | Tatcaaggaattaagagaagcaacatctccgaaagccaacaagga |
| | | aatcctcgatgtGAGTTTCTGCTTTGCTGTGTGG |
| 7 | Exemplary product after | ACACGACGCTCTTCCGATCTNNNNNNNNN |
| | amplicon completion (in | NTGCCAGTTAACGTCTTCCTTCTCtctctgtcata |
| | two steps) | gggactctggatcccagaaggtgagaaagttaaaattcccgtcgcta |
| | | tcaaggaattaagagaagcaacatctccgaaagccaacaaggaaat |
| | | cctcgatgtGAGTTTCTGCTTTGCTGTGTGGNN |
| | | NNNNNNNNAGATCGGAAGAGCACACGTC |
| 8 | Exemplary final product | AATGATACGGCGACCACCGAGATCTACAC |
| | | CTAGCGCTACACTCTTTCCCTACACGACGC |
| | | TCTTCCGATCTNNNNNNNNNNTGCCAGTTA |
| | | ACGTCTTCCTTCTCtctctgtcatagggactctggatccca |
| | | gaaggtgagaaagttaaaattcccgtcgctatcaaggaattaagagaa |
| | | gcaacatctccgaaagccaacaaggaaatcctcgatgtGAGTTT |
| | | CTGCTTTGCTGTGTGGNNNNNNNNNNAGA |
| | | TCGGAAGAGCACACGTCTGAACTCCAGTC |
| | | ACCCGCGGTTATCTCGTATGCCGTCTTCTG |
| | | CTTG |

1. A method of detecting genomic alterations within a biological sample comprising nucleic acids, comprising the steps of:
(a) extracting nucleic acid from the biological sample;
(b) performing a plurality of multiplexed PCR reactions on the extracted nucleic acid using:
a plurality of target capture primer pairs specific to a plurality of target genes that are capable of undergoing genomic alteration,
wherein each target capture primer pair comprises a forward target capture primer and a reverse target capture primer specific to a target gene that is capable of undergoing genomic alteration,
wherein each forward target capture primer and each reverse target capture primer comprises a target-specific sequence at its 3′ end that is specific to the target gene, a barcode sequence linker, and an adapter-specific sequence at its 5′ end,
thereby generating a plurality of amplicons comprising a plurality of target gene sequences corresponding to the plurality of target genes that are capable of undergoing genomic alteration;
(c) removing target capture primers that have not been incorporated into the plurality of amplicons generated in step (b) using at least one nuclease, thereby generating a purified mixture of the plurality of amplicons comprising the plurality of target gene sequences;
(d) amplifying the plurality of amplicons in the purified mixture obtained from step (c) by using universal indexed adapter primers to generate a sequencing library, wherein each amplicon of the sequencing library comprises two barcode sequences;
(e) purifying the sequencing library obtained from step (d);
(f) subjecting the purified sequencing library from step (e) to multiplex sequencing on a next-generation sequencing platform to obtain a plurality of sequencing reads;
(g) mapping the plurality of sequencing reads obtained from step (f) to a first reference genome;
(h) grouping the sequencing reads where the barcode sequences of the sequencing reads are identical into a consensus cluster;
(i) performing a sequence alignment of each consensus cluster obtained from step (h) with all consensus clusters having at least one overlapping barcode sequence, then determining the presence of a consensus base in each sequence alignment result to generate a consensus sequencing read;
(j) mapping each of the consensus sequencing read from step (i) with a second reference genome;
(k) identifying the differences between the sequencing read and the reference genome from step (j) to thereby determine the presence of genomic alteration in the nucleic acid present in the biological sample.

2. The method of claim 1, wherein the genomic alteration is selected from the group consisting of single-nucleotide variations, insertions, deletions, genomic copy number alterations, deletions of homopolymeric regions, total mutation or variant load, detection of microbial nucleic acid sequences, detection of polymorphisms or single-nucleotide variations in nucleic acid sequences, and DNA structural rearrangements giving rise to variant RNA molecules, wherein optionally the variant RNA molecules are selected from the group consisting of structural variants of RNA molecules and copy number alterations of RNA molecules.

3. The method of claim 1, wherein the nucleic acid is selected from the group consisting of DNA and RNA, wherein optionally the DNA is genomic DNA, tumor tissue DNA, circular DNA, circulating tumor DNA (ctDNA) or cell-free DNA (cfDNA), and wherein optionally the RNA is messenger RNA, circular RNA or non-coding RNA.

4. The method of claim 1, wherein the biological sample is selected from the group consisting of a liquid sample, a tissue sample, and a cell sample.

5. The method of claim 4, wherein the liquid sample is a bodily fluid, wherein optionally the bodily fluid is selected from the group consisting of blood, bone marrow, cerebral spinal fluid, peritoneal fluid, pleural fluid, lymph fluid, ascites, serous fluid, sputum, lacrimal fluid, stool, urine, saliva, ductal fluid from breast, gastric juice and pancreatic juice, wherein optionally the bodily fluid is blood, and wherein optionally the blood is plasma.

6. The method of claim 1, wherein the at least one nuclease used to remove the target capture primers that have not been incorporated into amplicons in step (c) is selected from the group consisting of:
(i) an endonuclease, wherein optionally the endonuclease is selected from the group consisting of mung bean nuclease, nuclease P1, nuclease S1 and combinations thereof; and
(ii) an exonuclease, wherein optionally the exonuclease is selected from the group consisting of exonuclease I, exonuclease T, exonuclease VII and combinations thereof;
wherein optionally the at least one nuclease comprises a combination of exonuclease I and exonuclease T.

7. The method of claim 1, wherein the sequencing library generated in step (d) comprises at least 1 amplicon, or at least 50 amplicons, or at least 100 amplicons, or at least 200 amplicons, or at least 300 amplicons, or at least 400 amplicons, or at least 500 amplicons, or at least 600 amplicons, or at least 700 amplicons, or at least 800 amplicons, or at least 900 amplicons, or at least 1000 amplicons, or at least 1100 amplicons, or at least 1200 amplicons, or at least 1300 amplicons, or at least 1400 amplicons, or at least 1500 amplicons, or at least 1600 amplicons, or at least 1700 amplicons, or at least 1800 amplicons, or at least 1900 amplicons, or at least 2000 amplicons, or at least 2100 amplicons, or at least 2200 amplicons, or at least 2300 amplicons, or at least 2400 amplicons, or at least 2500 amplicons, or at least 2600 amplicons, or at least 2700 amplicons, or at least 2800 amplicons, or at least 2900 amplicons, or at least 3000 amplicons, wherein optionally the sequencing library generated in step (d) comprises at least 2600 amplicons.

8. The method of claim 1, wherein only amplicons comprising barcode sequences and adapters on both their 5′ and 3′ ends from step (d) are subjected to multiplex sequencing on a next-generation sequencing platform as defined in step (f).

9. The method of claim 1, wherein the length of the target-specific sequence is from 15 nucleotides to 70 nucleotides, or from 16 nucleotides to 69 nucleotides, or from 17 nucleotides to 68 nucleotides, or from 18 nucleotides to 67 nucleotides, or from 19 nucleotides to 66 nucleotides, or from 20 nucleotides to 65 nucleotides, or from 21 nucleotides to 64 nucleotides, or from 22 nucleotides to 63 nucleotides, or from 23 nucleotides to 62 nucleotides, or from 24 nucleotides to 61 nucleotides, or from 25 nucleotides to 60 nucleotides, or from 26 nucleotides to 59 nucleotides, or from 27 nucleotides to 58 nucleotides, or from 28 nucleotides to 57 nucleotides, or from 29 nucleotides to 56 nucleotides, or from 30 nucleotides to 55 nucleotides, or from 31 nucleotides to 54 nucleotides, or from 32 nucleotides to 53 nucleotides, or from 33 nucleotides to 52 nucleotides, or from 34 nucleotides to 51 nucleotides, or from 35 nucleotides to 50 nucleotides, or from 36 nucleotides to 49 nucleotides, or from 37 nucleotides to 48 nucleotides, or from 38 nucleotides to 47 nucleotides, or from 39 nucleotides to 46 nucleotides, or from 40 nucleotides to 45 nucleotides, or from 41 nucleotides to 44 nucleotides, or 15 nucleotides, or 16 nucleotides, or 17 nucleotides, or 18 nucleotides, or 19 nucleotides, or 20 nucleotides, or 21 nucleotides, or 22 nucleotides, or 23 nucleotides, or 24 nucleotides, or 25 nucleotides, or 26 nucleotides, or 27 nucleotides, or 28 nucleotides, or 29 nucleotides, or 30 nucleotides, or 31 nucleotides, or 32 nucleotides, or 33 nucleotides, or 34 nucleotides, or 35 nucleotides, or 36 nucleotides, or 37 nucleotides, or 38 nucleotides, or 39 nucleotides, or 40 nucleotides, or 41 nucleotides, or 42 nucleotides, or 43 nucleotides, or 44 nucleotides, or 45 nucleotides, or 46 nucleotides, or 47 nucleotides, or 48 nucleotides, or 49 nucleotides, or 50 nucleotides, or 51 nucleotides, or 52 nucleotides, or 53 nucleotides, or 54 nucleotides, or 55 nucleotides, or 56 nucleotides, or 57 nucleotides, or 58 nucleotides, or 59 nucleotides, or 60 nucleotides, or 61 nucleotides, or 62 nucleotides, or 63 nucleotides, or 64 nucleotides, or 65 nucleotides, or 66 nucleotides, or 67 nucleotides, or 68 nucleotides, or 69 nucleotides, or 70 nucleotides, wherein optionally the length of the target-specific sequence is from 17 nucleotides to 40 nucleotides.

10. The method of claim 1, wherein the barcode sequence is an oligonucleotide comprising 8 to 30 random nucleotides.

11. The method of claim 1, wherein the barcode sequence is an oligonucleotide comprising 10 random nucleotides.

12. The method of claim 1, wherein the amount of nucleic acid within the biological sample is from 1 ng to 300 ng, or from 10 ng to 290 ng, or from 20 ng to 280 ng, or from 30 ng to 270 ng, or from 40 ng to 260 ng, or from 50 ng to 250 ng, or from 60 ng to 240 ng, or from 70 ng to 230 ng, or from 80 ng to 220 ng, or from 90 ng to 210 ng, or from 100 ng to 200 ng, or from 110 ng to 190 ng, or from 120 ng to 180 ng, or from 130 ng to 170 ng, or from 140 ng to 160 ng, or about 1 ng, or about 2 ng, or about 2.5 ng, or about 5 ng, or about 10 ng, or about 20 ng, or about 30 ng, or about 40 ng, or about 50 ng, or about 60 ng, or about 70 ng, or about 80 ng, or about 90 ng, or about 100 ng, or about 110 ng, or about 120 ng, or about 130 ng, or about 140 ng, or about 150 ng, or about 160 ng, or about 170 ng, or about 180 ng, or about 190 ng, or about 200 ng, or about 210 ng, or about 220 ng, or about 230 ng, or about 240 ng, or about 250 ng, or about 260 ng, or about 270 ng, or about 280 ng, or about 290 ng, or about 300 ng.

13. The method of claim 1, wherein the plurality of multiplexed PCR reactions performed on the nucleic acid in step (b) comprises a first PCR step comprising 3 to 15 PCR cycles and the amplification of the plurality of amplicons in the purified mixture as defined in step (d) comprises a second PCR step comprising 4 to 32 PCR cycles; wherein optionally the first PCR step comprises 3 to 5 PCR cycles; wherein optionally the second PCR step comprises 14 to 16 PCR cycles; and wherein the number of PCR cycles in the first PCR step and the second PCR step are independent of each other.

14. The method of claim 1, wherein the mapping in steps (g) and (j) and the sequence alignment in step (i) are performed using bioinformatic algorithms, wherein optionally the algorithm used for the mapping in steps (g) and (j) is bwa-mem, and wherein optionally the algorithm used for the sequence alignment in step (i) is MAFFT.

15. The method of claim 1, wherein each consensus cluster as defined in step (h) comprises at least 1 family member.

16. The method of claim 1, wherein prior to performing step (i), consensus clusters with fewer than 5 family members are removed from downstream analysis, wherein optionally consensus clusters with fewer than 4 family members are removed from downstream analysis, or consensus clusters with fewer than 3 family members are removed from downstream analysis, or consensus clusters with fewer than 2 family members are removed from downstream analysis.

17. The method of claim 1, wherein the first reference genome as defined in step (g) and the second reference genome as defined in step j) comprise identical gene sequences.

18. The method of claim 1, wherein prior to performing step (g), (I) bases having poor quality scores are removed, and (II) universal indexed adapter primers and barcode sequences are trimmed.

19. The method of claim 1, wherein the biological sample is obtained from a subject having and/or suspected of having a disease, and wherein optionally the disease is cancer or an infectious disease.

20. The method of claim 19, wherein the cancer is selected from the group consisting of leukemia, lung cancer, colorectal cancer, breast cancer, pancreatic cancer, prostate cancer, nasopharyngeal cancer, liver cancer, cholangiocarcinoma, esophageal cancer, urothelial cancer, and gastrointestinal cancer.

21. The method of claim 19, wherein the infectious disease is viral infection or bacterial infection.

22. A kit for detecting genomic alterations within a biological sample according to the method of claim 1, comprising a plurality of target capture primer pairs specific to a plurality of target genes that are capable of undergoing genomic alteration as defined in claim 1(b), and instructions for use in the method.

23. The kit according to claim 22, wherein the kit further comprises:
a buffer for performing a plurality of multiplexed PCR reactions,
a DNA polymerase,
a plurality of deoxynucleoside triphosphates (dNTPs), and
one or more nuclease capable of removing unincorporated target capture primers.
